(19) United States
(12) Patent Application Publication
(10) Pub. No.: US 2005/0031679 A1
(43) Pub. Date: Feb. 10, 2005

(54) Method for producing liposomal formulations of active ingredients

(76) Inventors: Clemens Unger, Freiburg (DE); Ulrich Massing, Freiburg (DE); Regina Moog, Hufingen (DE)

Correspondence Address: FULBRIGHT & JAWORSKI, LLP, 666 FIFTH AVE, NEW YORK, NY, 10103-3198, US

(21) Appl. No.: 10/937,556

(22) Filed: Sep. 9, 2004

Related U.S. Application Data
(63) Continuation of application No. 09/646,030, filed on Nov. 14, 2000.

(30) Foreign Application Priority Data
Mar. 27, 1998 (DE) 198 13 773.7

(51) Int. Cl. A61K 9/127

(52) U.S. Cl. 424/450

(57) ABSTRACT

The invention relates to a method for producing liposomes which comprise as active ingredient at least one physiologically active or/and diagnostic compound, by production of a mixture of membrane-forming amphiphiles, dispersion of the mixture in water and high pressure homogenization of the dispersion until the liposomes are formed, wherein (a) the mixture is adjusted to result in a liposome preparation containing at least 20% by weight lipids, (b) the active ingredient is added to this liposome preparation and (c) the active ingredient-containing mixture is incubated with mechanical agitation until at least 10% of the liposomes have a content of the active ingredient, the temperature being controlled so that negligible phospholipid hydrolysis occurs, but diffusion is increased. The invention also relates to the liposome preparation produced by this method and to the use of this liposome preparation as diagnostic or therapeutic composition, which, where appropriate, contains further excipients, carriers or/and stabilizers.

The invention relates to a method for producing liposomes which comprise as active ingredient at least one physiologically active or/and diagnostic compound, and to the liposome preparation produced by this process and to the use of the liposome preparation as diagnostic or therapeutic composition.

Liposomes are spherical structures composed of amphiphiles. The liposomes are produced in aqueous solutions by self-aggregation of the amphiphiles, there being formation of a lipid bilayer which encloses an aqueous interior.

Depending on physical parameters such as pressure, temperature and ion concentration, and the lipids and additives available, there is formation of unilamellar, oligolammellar or multilamellar liposomes. The liposomes may carry a positive or negative excess charge depending on their ingredients.

Liposomes can also be loaded with active ingredients which, depending on the lipophilicity or hydrophilicity, are enclosed in the lipid layer or in the aqueous interior of the liposomes. Liposomes of this type are used in diagnostic detection methods or as therapeutic compositions for transporting active ingredients in the body or as active ingredient depot.

The properties of the liposomes such as, for example, their stability or storability are essentially determined by the substances present in the lipid layer.

Liposomes are synthesized using membrane-forming lipids such as, for example, phosphatidylcholine, phosphatidylglycerol, phosphatidylinositol, phosphatidylserine and phosphatidylethanolamine, sphingomyelin, membrane-forming amphiphiles such as, for example, block polymers, alkyl esters, ethers, amides of alcohols, diols and polyols, amines, amino acids, peptides and sugars and cholesterol, and other substances (cf. F. Szoka Jr. and D. Papahadjopoulos, Comparative Properties and Methods of Preparation of Lipid Vesicles (Liposomes), Ann. Rev. Biophys. Bioeng. 1980, 9:467-508; Liposomes: From Physical Structure To Therapeutic Application (1981), Knight, C. G. (ED.), Elsevier, North Holland Biomedical Press, chapter 2: H. Eibel, Phospholipid Synthesis, 19-50; chapter 3: F. Szoka and D. Papahadjopoulos, Liposomes: Preparation and Characterization, 51-104).

Mechanical methods are known for producing active ingredient-containing liposomes and entail treating a dispersion comprising the membrane ingredients and active ingredients by the action of strong mechanical forces, for example high pressure homogenization, for example using a French press. Another method is based on the replacement of the organic solvent by aqueous medium or on simple removal of the detergent present in the dispersion. Another method for producing liposomes is based on changing the pH (cf. F. Szoka Jr. and D. Papahadjopoulos).

Because of the properties of the active ingreients to be enclosed, it is in many cases necessary for producing active ingredient-containing liposomes to complete the liposome formation and inclusion of the active ingredients in only one step of the method. The liposome formation and inclusion of active ingredients in one step is, however, often impracticable and may involve various disadvantages: thus, for example, under the conditions of high pressure homogenization (French press, Gaulin . . . ) there may be, due to the interactions between the active ingredient and the lipids employed and the medium, decomposition of the active ingredient, hydrolysis of the lipids or both. For the same reasons, sterilization of the active ingredient-containing formulation is often impossible; lipid hydrolysis and/or decomposition of the active ingredient are observed. An alternative sterile filtration for sterilization is not possible for producing liposome gels because their viscosity is too high. Sterile filtration is often also impossible for liposome dispersions because of the size of the vesicles.

In addition, designing a particular liposome gel or a liposome dispersion without simultaneous addition of the active ingredient is impossible because active ingredients often crucially-influence the formation of the lipid bilayer and thus may also have an influence on the size of the resulting vesicles.

Owing to the apparatus required for producing liposomes, it is often necessary to produce a minimum quantity of a liposomal formulation far exceeding the quantity actually necessary for investigating the preparation. This is a disadvantage in particular when the active ingredient used for inclusion is available only in limited quantity or is very costly.

Thus, only active ingredient-containing liposomes can be investigated for their characteristics. If the liposome preparation produced in this way does not show the required properties it is necessary to discard all this preparation, including the costly active ingredients present therein. This is a disadvantage in particular when the active ingredient used for the encapsulation is available only in limited quantity or is very costly.

It is additionally necessary for particular applications of liposome preparations, in particular on therapeutic use, to sterilize the liposome preparation. Generally used for this purpose is sterilization by autoclaving, in which elevated temperature and pressure conditions occur. However, certain substances enclosed in liposomes bring about phospholipid hydrolysis at elevated temperature, so that decomposition of the liposome preparation occurs with such a treatment. In addition, the active ingredients may be unstable at elevated temperature and decompose on autoclaving. Thus, sterilization by autoclaving cannot be used for a liposome preparation of this type.

Another disadvantage in the production of an active ingredient-containing liposome formulation by high pressure homogenization is the possible formation of aerosols, which represents a considerable hazard for the attendant staff on use of certain active ingredients. The necessary safety precautions are elaborate and make the production process difficult. This leads to a greater expenditure of time., increased use of working materials and a greater staff requirement, which considerably increase the production costs.

The object on which the invention is based was thus to provide a method for producing active ingredient-containing liposomes with which sterile liposome preparations can be provided at low cost and without hazard for the attendant staff, and the disadvantages of the prior art can be at least partly improved.

This object is achieved by a method for producing liposomes which comprise as active ingredient at least one physiologically active or/and diagnostic compound, by production of a mixture of membrane-forming amphiphiles, dispersion of the mixture in water and high pressure homogenization of the dispersion until the liposomes are formed, which comprises
(a) adding the active ingredient to any liposome preparation, preferably a liposome gel, which contains at least 20% by weight lipid, and
(b) incubating the active ingredient-containing mixture, where appropriate with mechanical agitation, until
   (i) hydrophilic active ingredients are distributed in the liposomes and between the liposomes in accordance with the proportions by volume of aqueous medium inside and outside the liposomes in the preparation (equilibrium state) or
   (ii) lipophilic compounds are incorporated in the lipid bilayer, or
   (iii) amphiphilic compounds have surmounted the lipid bilayer and are incorporated or associated in the outer regions of the lipid bilayer.

During this the temperature is controlled so that negligible lipid hydrolysis occurs, but diffusion of the active ingredients across the lipid bilayer or uptake in the bilayer is increased.

It is possible to employ as active ingredients all compounds which, under the given conditions, are able to diffuse across lipid bilayers (hydrophilic and amphiphilic compounds) or be incorporated in lipid bilayers (lipophilic compounds).

It was surprising that it is possible with the method according to the invention to load a finished liposome preparation subsequently with active ingredients with good yields. It was unexpected that the active ingredient can be added as solid or in concentrated aqueous solution to the finished liposome preparation, and increasing the temperature and, where appropriate, simple mechanical mixing, stirring or shaking, using an apparatus suitable for this purpose, such as, for example, a horizontal shaker, leads to good rates of inclusion of the active ingredient in the liposome preparation. Another advantage is that the method according to the invention can be carried out straightforwardly, and the requirements for equipment and staff are low. With the method according to the invention it is also possible to encapsulate toxic substances without special safety measures and the use of elaborate safety precautions in terms of equipment. In contrast to known methods, a hazard for the attendant staff due to formation of aerosols is substantially precluded in the method according to the invention. The method according to the invention thus leads to a considerable reduction in costs and simplification of the process for producing active ingredient-containing liposome preparations.

It is possible to use in the process according to the invention all known liposome preparations such as, for example, unilamellar, bilamellar, multilamellar liposome preparation or/and liposomes with positive or negative excess charge.

The ingredients which can be used for the lipid layer of the liposomes are all membrane-forming amphiphiles suitable for this purpose, besides other substances, such as, for example, phosphatidylcholine, phosphatidylglycerol, phosphatidylinositol, phosphatidylserine, phosphatidylethanolamine, sphingomyelin, cholesterol, saturated and unsaturated fatty acids, membrane-forming amphiphiles such as, for example, block polymers, alkyl esters, ethers, amides of alcohols, dials, polyols, amines, amino acids, peptides and sugars, and other substances which are disposed in the lipid layer in an aqueous environment. It is possible where appropriate for organic or inorganic salts, and acids or bases for adjusting the pH and the osmotic pressure, to be dissolved in the liposome preparation. Especially on use of the liposome preparation produced by the method according to the invention for medical purposes, such as, for example, injection or introduction of the liposome preparation according to the invention into body cavities there is use of sterile solutions and substances, and water complying with the German Pharmacopeia (“Water for Injections”). The liposome preparation can be sterilized before incubation with the active ingredient by suitable methods, such as, for example, autoclaving.

The liposomes of the liposome preparation may have a small, intermediate or large diameter; a liposome preparation with liposomes having an average diameter of 10 to 400 nm is preferably used.

The liposome preparation may be a liposome-containing dispersion; a gel composed of liposomes is preferably used.

It is possible to use for the method according to the invention polar and amphiphilic substances, and substances with diagnostic and therapeutic activity, which are able, because of their diffusion properties, to diffuse (slowly) into or across the lipid bilayer. Examples are, for example cytostatics, antibiotics, contrast agents. It is possible to enclose a single active ingredient or else a plurality thereof. Enclosure in the interior of the liposomes or/and in the membrane or/and between the membrane layers is possible. All these modes of inclusion are referred to comprehensively as incorporation:

In a preferred embodiment, the therapeutic active ingredient used is gemcitabine, vincristine, amphotericin b or/and anthracyclines, in particular doxorubicin. Calcein is preferably used as a fluorescent dye employed in research.

The conditions for the method are adapted to the particular liposome preparation used and active ingredient used, in order to obtain the required uptake of the active ingredient in the liposome preparation. The rate of uptake and efficiency of inclusion of the active ingredient in the liposome preparation are influenced by the diffusion properties of the active ingredient and the characteristics of the liposomes.

The diffusion properties of the active ingredient in turn depend on parameters such as, for example, molecular weight, oil/water coefficient or degree of protonation.

The liposome characteristics are determined by parameters such as, for example, nature and content of the membrane-forming amphiphiles, content of cholesterol, and the content and nature of additional substances in the lipid layer. In particular, the maximum ratio of aqueous phase in the liposomes to aqueous phase outside the liposomes, and the presence of the liposomes in a liposome dispersion or in a liposome gel, determine the efficiency of inclusion.

All the parameters relevant for the method are adapted to be mutually optimal. In particular, the temperature is adjusted so that diffusion of the active ingredient into the liposomes is increased with negligible phospholipid hydrolysis occurring. The active ingredient-containing mixture is preferably incubated at a temperature of 30 to 80° C., more preferably of 50-70° C. and most preferably about 60° C. If the lipid mixture shows a phase transition temperature above room or storage temperature it is preferred to heat above this temperature.

The incubation temperature and incubation time are mutually dependent, i.e. on increasing the incubation temperature it is possible to reduce the incubation time if it is wished to obtain the same degree of inclusion as at a reduced incubation temperature.

The individual components are incubated until the maximum uptake is reached (equilibrium state for hydrophilic compounds, virtually complete uptake for lipophilic compounds). Thus, if there is incomplete uptake of the active ingredient in the liposomes of the gel, part of the active ingredient is present in the liposomes and the remaining active ingredient added to the liposome preparation is present outside the liposomes in the suspension or the gel. A liposome gel produced in this way may release the active ingredient in a delayed manner over a lengthy period, and a good release-slowing action is achieved due to the fact that an active ingredient must, in order to leave the liposome gel, often diffuse out of individual liposomes and diffuse back into liposomes, because the liposomes are very tightly packed. On the other hand, with liposome dispersions produced in the novel way it is possible for the active ingredient not enclosed in the liposomes to have a rapid effect (initial dose), while the enclosed portion is released and has an effect with a delay. Alternatively, the unincorporated part of the active ingredient is removed from the liposome preparation if free active ingredient is not required. An active ingredient-containing liposome gel produced according to the invention is preferably used for medical applications.

As described above, the rate of uptake and efficiency of inclusion depend inter alia on the properties of the active ingredient. Lipophilic and amphiphilic active ingredients are essentially enclosed in the lipid layer of the liposomes, whereas hydrophilic active ingredients are essentially enclosed in the aqueous interior of the liposomes. Lipophilic active ingredients show very high rates of inclusion. They may be incorporated in liposomes with a rate of inclusion of up to 100%. Amphiphilic compounds which are incorporated or attached on and in the outer membrane areas lead to rates of inclusion of up to 50%. The rate of inclusion may be higher with liposomes composed of a plurality of bilayers. The mixture is preferably incubated until the lipophilic active ingredient has been completely taken up in the liposome membrane.

A further aspect of the invention is a liposome preparation obtained or obtainable by a method as described above.

Yet a further aspect of the invention is the use of a liposome preparation obtained or obtainable by a method as described above as diagnostic or therapeutic composition which comprises, where appropriate, further excipients, carriers or/and stabilizers such as, for example, sugars, reduced sugars and polyalcohols.

A further aspect of the invention is a liposome preparation obtained or obtainable by redispersion of the liposomes by stepwise addition of aqueous medium to the liposome-containing gel. The formulation obtained in this way can be administered systemically, for example by i.v. injection. Unenclosed active ingredient can be removed by suitable methods.

The following examples are intended to explain the invention further.

### EXAMPLES

### Example 1

A liposome gel which contains the cytostatic gemcitabine as active component cannot be autoclaved because this antitumor agent promotes the hydrolysis of the phospholipids, leading to complete phospholipid degradation under standard conditions during autoclaving. The requirement for sterility of drug products for parenteral use can be achieved only by subsequent incorporation of the active ingredient into the liposome gel. This is done by autoclaving the liposome gel and a highly concentrated, buffered gemcitabine solution separately and then mixing under aseptic conditions. After incubation at 60° C. for four hours, an efficiency of inclusion of more than 30% is reached (equilibrium).

This preparation is suitable for direct instillation into the abdominal cavity in cases of ovarian carcinoma, for bladder irrigation in cases of bladder carcinoma and for intratumoral administration, and for i.v. administration after redispersion.

### Example 2

The cytostatic vincristine is highly effective and is employed clinically in low total doses. Strict safety measures are necessary during the high pressure homogenization on inclusion of the active ingredient by the conventional technique for producing liposome gels, in order to prevent inhalation of resulting aerosols and possible late effects on the attendant staff. In addition, vincristine is unstable under the conditions of high pressure homogenization or autoclaving. Subsequent incorporation of the substance considerably reduces the formation of aerosols and thus significantly reduces the working risk. Production takes place as in Example 1.

This formulation can also be employed for local treatment of pulmonary carcinomas and can be administered i.v. after redispersion.

### Example 3

The fluorescent marker calcein is frequently employed in fundamental research. It cannot be autoclaved, so that sterile calcein liposome gels can be produced only by subsequent addition of calcein. Production takes place as in Example 1 after sterile filtration of the calcein solution. This preparation can be used for release and distribution tests, for example in microbiology.

### Example 4

The method is also suitable in principle for encapsulating lipophilic substances which are enclosed not in the aqueous compartment but in the liposome layer. For this purpose, the antibiotic amphotericin B is added as solid substance to the liposome gel and then incubated with shaking at 50° C. for 3 hours. The efficiency of inclusion is 95%.

1-15 (canceled)

16. A method for producing liposomes, comprising gemcitabine as active ingredient, by producing a mixture of membrane-forming amphiphiles, dispersing said mixture in water and high pressure homogenisation of the dispersion until the liposomes are formed, wherein the active ingredient gemcitabine is added to the liposomes and the active ingredient-comprising mixture is incubated until hydrophilic active ingredients are distributed in the liposomes and between the liposomes in accordance with the proportions by volume of aqueous medium inside and outside the liposomes in the preparation (equilibrium state).

17. The method as claimed in claim 16, wherein a gel formed of liposomes is used as lipid-comprising liposome preparation.

18. The method as claimed in claim 17, wherein a liposome gel with a lipid content of at least 20% by weight is used.

19. The method as claimed in claim 16, wherein the incubation is carried out during mechanical agitation.

20. The method as claimed in claim 17, wherein the incubation is carried out during mechanical agitation.

21. The method as claimed in claim 16, wherein the incubation is carried out during mechanical agitation.

22. The method as claimed in claim 16, wherein a liposome-comprising dispersion is used.

23. The method of claim 17, wherein the liposomes are re-dispersed by adding aqueous medium step by step to the liposome-comprising gel.

24. The method of claim 18, wherein the liposomes are re-dispersed by adding aqueous medium step by step to the liposome-comprising gel.

25. The method of claim 22, for the production of a formulation which can be administered systemically.

26. The method of claim 23, for the production of a formulation which can be administered systemically.

27. The method of claim 16, wherein the active ingredient-comprising mixture is incubated at a temperature of 30 to 80° C.

28. The method of claim 27, wherein said mixture is incubated at a temperature of from 50 to 70° C.

29. The method of claim 27, wherein said mixture is incubated at 60° C.

30. A liposome preparation obtained according to a method of from 50 to 70° C.

31. A composition for producing a liposome preparation according to claim 22 comprising component a) liposomes produced by high pressure homogenisation; and
component b) gemcitabine.

32. The composition as claimed in claim 25, wherein components a and b are autoclaved.

33. The composition as claimed in claim 31, wherein component b comprises a buffered gemcitabine solution.

34. The composition as claimed in claim 32, wherein component b comprises a buffered gemcitabine solution.
